(19) United States
(12) Patent Application Publication
(10) Pub. No.: US 2013/0296758 A1
(43) Pub. Date: Nov. 7, 2013

(54) ROLL-UP KNEE BRACE LINER

(71) Applicant: David J. Castillo, Lake Forest, CA (US)

(72) Inventor: David J. Castillo, Lake Forest, CA (US)

(21) Appl. No.: 13/464,058

(22) Filed: May 4, 2012

(51) Int. Cl. A61F 5/01

(52) U.S. Cl. CPC A61F 5/0123
USPC 602/26

(57) ABSTRACT

Provided is a brace liner configured for use with a conventional knee brace, wherein the brace liner is configured to include a portion which is worn under the knee brace and a portion that is folded over the knee brace to define a pocket which captures the knee brace to hold the knee brace in proper position on the user's knee. The brace liner is worn like a conventional pair of pants, and thus is supported by the wearer's hips, which in turn supports the knee brace from the wearer's hips, to prevent the knee brace from migrating downwardly during usage of the brace. The brace liner also tightly conforms to the brace to reduce the appearance of the knee brace.

### CROSS-REFERENCE TO RELATED APPLICATIONS

Not Applicable.

### STATEMENT RE: FEDERALLY SPONSORED RESEARCH/DEVELOPMENT

Not Applicable

### BACKGROUND OF THE INVENTION

1. Technical Field of the Invention

The present invention relates generally to a liner for a knee brace, and more specifically, a liner supported by the hips of a wearer, wherein the liner is configured to hold the knee brace in proper position on the wearer's knee and to provide a smooth outer contour around the knee brace.

2. Description of the Related Art

Knee braces are well known in the art and are designed to protect a wearer's knee joint from injury, particularly during physical activities, such as motorcycle racing, skiing, football, basketball, and the like. In general, knee braces limit the range of motion of the knee joint to a safe range of motion, i.e., knee braces restrict the joint from movement which may otherwise cause ligament tears, meniscus tears, hyperextension, etc.

Conventional knee braces generally include an upper frame member, a lower frame member, and a pivot member pivotally connecting the upper frame member to a lower frame member. The upper and lower frame members are pivotable relative to the pivot member to define the pivotal range of motion of the knee joint.

The knee brace is generally positionable on the wearer's leg about the knee, such that the upper frame member extends slightly above the wearer's knee along the wearer's thigh and the lower frame member extends slightly below the wearer's knee along the wearer's shin. Operation of the knee brace is typically most effective when the brace is properly positioned on the wearer's knee, which in most cases means that the wearer's knee and the brace pivot about substantially the same axis.

However, one deficiency commonly associated with knee braces is that they tend to shift during usage thereof. For instance, over time, the weight of the knee brace may pull the brace down from its proper position. In addition, repeated flexion or movement of the wearer's leg may additional cause the brace to shift.

Another deficiency commonly associated with knee braces is the bulky appearance related thereto. A large knee brace worn by a user is readily identifiable due to its cumbersome appearance. Furthermore, the bulky nature of the knee brace may cause the brace to “catch” onto nearby objects. For instance, in motocross racing, a knee brace worn by a racer may “catch” onto an adjacent barrier, which may cause the racer to crash and lead to serious injury to the racer, as well as nearby racers and spectators.

Still another deficiency associated with conventional knee braces is that the inner surface of the knee brace is generally disposed in direct contact with the user's leg, which may be uncomfortable, particularly if the knee brace is worn for extended periods of time. As the user perspires, the perspiration may be soaked up by the knee brace, which makes it difficult to keep the knee brace clean.

Therefore, there is a need in the art for a device which addresses the deficiencies discussed above. In particular, there is a need for a device which properly positions a knee brace in the wearer's leg and keeps the brace in proper position during use. There is also a need for a device which compresses the brace to create a smooth external configuration to mitigate latching on to external objects.

### BRIEF SUMMARY OF THE INVENTION

The present invention specifically addresses and alleviates the above-identified deficiencies in the art. There is provided a brace liner configured for use with a conventional knee brace, wherein the brace liner is configured to include a portion which is worn under the knee brace and a portion that is folded over the knee brace to define a pocket which captures the knee brace to hold the knee brace in proper position on the user's knee. The brace liner is worn like a conventional pair of pants, and thus is supported by the wearer's hips, which in turn supports the knee brace from the wearer's hips to prevent the knee brace from migrating downwardly during usage of the brace. The brace liner also tightly conforms to the brace to reduce the appearance of the knee brace.

According to one embodiment, the brace liner includes a first limb portion having a first surface and an opposing second surface, with the first surface defining an opening sized to receive the user's limb. The brace liner additionally includes a first end portion, a second end portion, and a middle portion extending between the first end portion and the second end portion. The first limb portion is transitional between an unfolded configuration and a folded configuration, with the brace being disposable over the first limb portion when the first limb portion is in the unfolded configuration. The second end portion is folded toward the first end portion when the first limb portion transitions from the unfolded configuration toward the folded configuration to capture at least the distal end portion of the brace within the first limb portion.

The brace liner may additionally be configured such that the first limb portion defines an annular fold as the first limb portion transitions from the unfolded configuration toward the folded configuration.

The brace liner may additionally include a waist portion connected to the first limb portion, wherein the waist portion is wearable on the user's waist.

The brace liner may further include a slit formed within the first limb portion adjacent the second end portion and extending between the first surface and the second surface. The slit may be configured to allow the first limb portion to extend about the distal end portion of the brace as the first limb portion transitions into the folded configuration.

The present invention is best understood by reference to the following detailed description when read in conjunction with the accompanying drawings.

### BRIEF DESCRIPTION OF THE DRAWINGS

These as well as other features of the present invention will become more apparent upon reference to the drawings wherein:

FIG. 1 is a front view of an embodiment of a brace liner worn by a user under a pair of knee braces, wherein the brace liner is in an unfolded configuration;

FIG. 2 is a front view of the brace liner shown in FIG. 1, wherein the brace liner is in a folded configuration around the knee braces;

FIG. 3 is a rear view of the brace liner, wherein one leg portion of the liner is in the folded configuration and the other leg portion is in the unfolded configuration;

FIG. 4 is a front view of the brace liner depicted in FIG. 3;

FIG. 5 is a front view of the brace liner;

FIG. 6 is a side view of the brace liner; and

FIG. 7 is a rear view of the brace liner.

Common reference numerals are used throughout the drawings and detailed description to indicate like elements.

### DETAILED DESCRIPTION OF THE INVENTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

Referring now to the drawings, wherein the showings are for purposes of illustrating a preferred embodiment of the present invention and not for purposes of limiting the same, there is shown a brace liner 10 constructed in accordance with an embodiment of the present invention and configured for use with a conventional knee brace 12. The brace liner 10 is placed on a user similar to a conventional pair of pants. The knee brace 12 is then placed over the brace liner 10 and then the ends of the brace liner leg portions are folded around the knee brace 12 to hold the brace 12 in proper position and to support the brace 12 from the user's hips. In this regard, a portion of the brace liner 10 extends under the brace 12 while another portion of the liner 10 extends over the brace 12 when the liner 10 is folded to define a pocket which captures the brace 12 and holds the brace 12 in place. The brace liner 10 also compresses against the brace 12 to define a smooth external configuration which hides the appearance of the brace 12.

The brace liner 12 includes a pair of leg portions 14, 16 which are joined at a waist section 18. The waist section 18 includes an upper end portion 20 that extends around the wearer's hips. In this regard, the upper end portion 20 is sized to fit snuggly on the wearer to allow the brace liner 10 and brace 12 to be supported by the wearer's hips or waist. To this end, the waist section 18 may be adjustable to conform to the size of the wearer. For instance, the waist section 18 may include an elastic band which compresses against the wearer during use, but is also stretchable to facilitate donning and removal of the brace liner 12. It is also contemplated that other adjustment mechanisms, such as zippers, buttons, belts, and the like may also be used without departing from the spirit and scope of the present invention. Furthermore, the brace liner 12 may be sold in various sizes to accommodate all sizes of wearers.

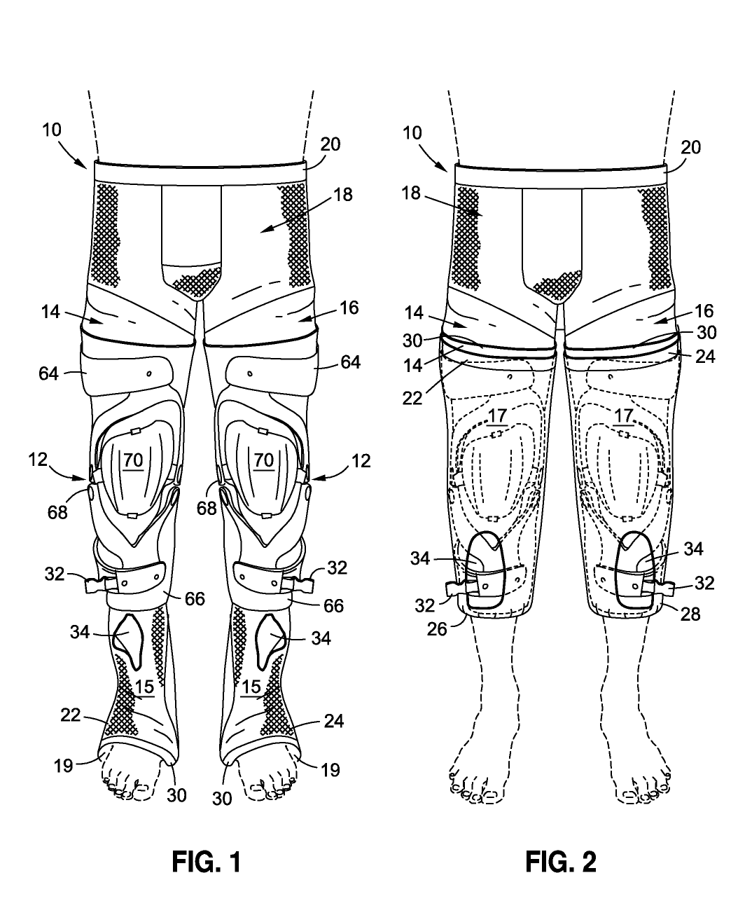

The leg portions 14, 16 extend from the waist section 18 similar to a conventional pair of pant legs and define a first surface 15 (See FIG. 1) and an opposing second surface 17 (See FIG. 2). The first surface 15 defines respective openings 19 through which the user's legs may extend through to allow the legs portions 14, 16 to cover the thigh, knee and calf of the wearer. Each leg portion 14, 16 extends from the waist section and terminates at a distal end portion 22, 24 thereof. According to one embodiment, the leg portions 14, 16 should define a length which extends at least to the wearer's ankles, and preferably past the ankles to cover a portion of the wearer's feet. The brace liner 10 may be sold in various sizes such that the size of the leg portions 14, 16 is commensurate with the size of the wearer's legs. As will be described in more detail below, the leg portions 14, 16 are transitional between an unfolded configuration, wherein the leg portion 14, 16 extends over the wearer's leg (see FIG. 1), and a folded configuration, wherein the distal end portion 22, 24 of the leg portion 14, 16 is folded upwardly over the knee brace 12 to cover the brace 12.

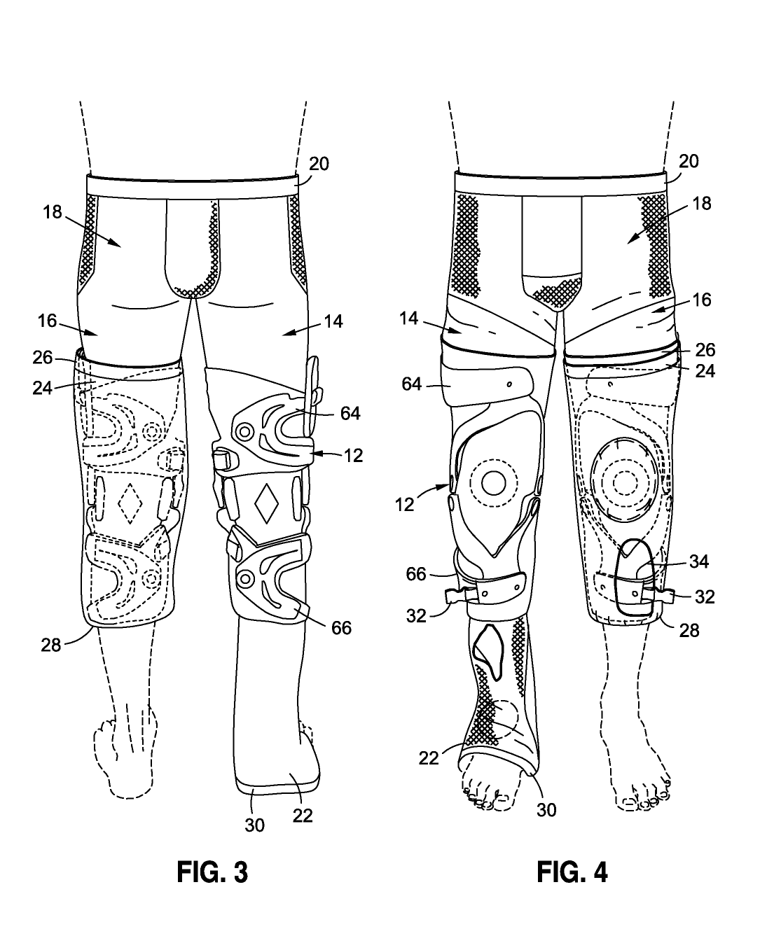

In use, the user dons the brace liner 10 similar to a pair of pants, such that the leg portions 14, 16 extend over the wearer's legs in the unfolded configuration and the waist portion 18 is supported by the midsection (i.e., waist or hips) of the wearer. Subsequently, the user places the knee brace 12 over the unfolded brace liner 10 on the wearer's knee. The knee brace 12 may be placed on the user's knee in a conventional manner. In this regard, the use of the brace liner 10 may not affect the manner in which the knee brace 10 is placed on the user's knee. Furthermore, it is understood that the brace liner 10 may be used by a user wearing a pair of knee braces 12, as shown in FIGS. 1-4 or a user wearing only a single knee brace 12 (i.e., a brace 12 either on the left knee or right knee). If the user is only using a single knee brace 12, it is contemplated that various embodiments of the liner 10 may be manufactured with only a single leg portion 14, 16, or a leg portion 14, 16 that is detachable from the waist section 18.

The next step is to fold the brace liner 10 over the knee brace(s) 12, i.e., transition the brace liner 10 from the unfolded configuration to the folded configuration. This is accomplished by grasping the distal end portion 22, 24 of the respective leg portion 14, 16 and pulling the distal end portion 22, 24 over the knee brace 12 until the brace liner 10 is taut. In this regard, when the distal end portion 22, 24 transitions from the unfolded configuration to the folded configuration, an annular fold line 26, 28 is formed within the leg portion 14, 16. The distal end portion 22, 24 is pulled until the respective fold line 26, 28 is adjacent the lower end portion of the respective knee brace 12. The distal end portion 22, 24 preferably remains in place adjacent the user's thigh when the brace liner 10 is in the folded configuration. Along these lines, the distal end portion 22, 24 may include an elastic band 26 which compresses against the user's thigh when the leg portion 14, 16 is transitioned into the folded configuration. The brace liner 10 may include other restraining members on the distal end portions 22, 24 and the external surfaces of the leg portions 14, 16, which hold the leg portions 14, 16 in the folded configuration. For instance, complimentary fasteners, such as hook and loop material, buttons, snaps, etc. may be used.

When the knee brace 10 is in the folded configuration, a portion of the brace liner 10 extends under the knee brace 10 and another portion of the brace liner 10 extends over the knee brace 12. In this regard, when the brace liner 10 is transitioned from the unfolded configuration to the folded configuration, the brace liner 10 forms a pocket around the knee brace 12 to hold the knee brace 12 in place on the user's leg. The brace liner 10 is supported by the wearer's hips/waist to prevent the brace liner 10 from migrating downwardly while the wearer uses the brace 12. Thus, the brace 12 remains in position within the pocket defined by the liner 10 to prevent the brace 12 from undesirably moving during usage. The fold line 26, 28 prevents the brace 12 from moving downwardly, while the tight compression of the liner 10 over the brace 12 mitigates rotation of the brace 12 along the user's leg.

The tight compression of the brace liner 10 over the brace 12 also defines a smooth external configuration. Thus, the bulky and rough appearance associated with the knee brace 12 is mitigated. Furthermore, the smooth external configuration protects against the knee brace 12 snagging onto an adjacent object, which enhances the safety of wearing the knee brace 12.

It is contemplated that the knee brace 12 worn by the wearer may include a boot connector 32 for connecting the knee brace 12 to a boot or similar footwear worn by the wearer. Such a boot connector 32 may be common on knee braces 12 adapted for use by moto-cross racers. The connection between the knee brace 12 and the boot may prevent the user's foot from over-rotating relative to the wearer's knee, which may otherwise cause knee ligament damage. For a more detailed discussion related to the connection between the knee brace 12 and the boot, please see refer to U.S. Pat. No. 7,458,949, entitled FLEXIBLE TETHER MEMBER CONNECTING A KNEE BRACE TO A BOOT, owned by Asterisk.Asterisk LLC, Applicant of the present patent application.

According to one embodiment, the brace liner 10 is configured to accommodate the boot connector 32 extending from the bottom of the knee brace 12. More specifically, the brace liner 10 may include a slit 34 formed within each leg portion 14, 16 through which the boot connector 32 may extend through when the brace liner 10 is placed in the folded configuration (see FIG. 2). As the line 10 transitions from the unfolded configuration toward the folded configuration, the slit 34 may allow the liner 10 to expand around the distal end portion of the brace 12. Thus, the slit 34 may be advantageous not only for braces 12 that include a connector 32 to allow the connector 32 to pass through the slit 34, but for most braces 12 to allow the liner 10 to stretch around the lower portion of the brace 12.

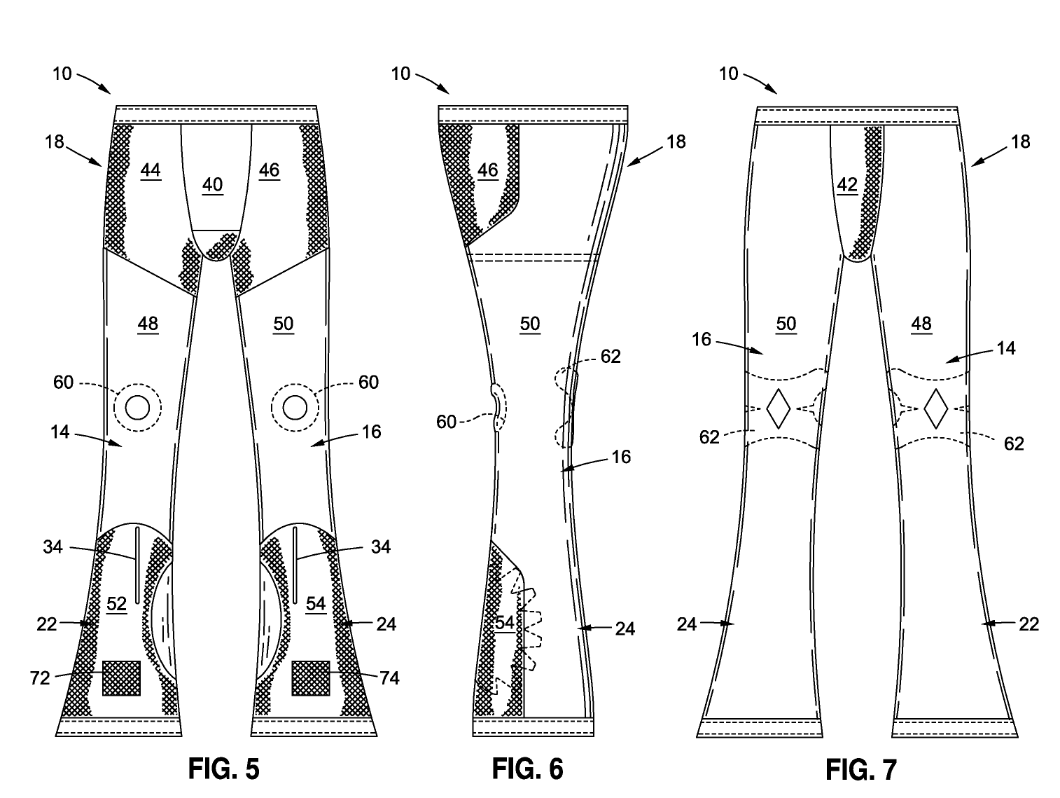

Referring now specifically to FIGS. 5-7, the brace liner 10 may be formed from several different materials to enhance the comfort and functionality of the brace liner 10. According to one embodiment, the waist section 18 of the brace liner 10 includes a crotch panel 40, a rear panel 42, and a pair of side panels 44, 46 extending between the crotch panel 40 and the rear panel 42. The crotch panel 40 may be formed from a moisture wicking elastic material, while the rear panel 42 and the side panels 44, 46 may be formed from a soft mesh material.

The leg portions 14, 16 may be comprised of respective primary leg panels 48, 50 which are joined to the side panels 44, 46 and the rear panel 42. The primary leg panels 48, 50 may be formed from a moisture wicking elastic material. The front and sides of each distal end portion 22, 24 may include a secondary leg panel 52, 54 and an inner leg panel 56, 58. The secondary leg panels 52, 54 may be formed from a soft mesh material and the inner leg panels 56, 58 may be formed from a thin leather material. The thin leather material may provide a strong durable material which resides between the knee brace 12 and the motorcycle, when the brace 12 is worn by a motorcycle rider and the brace liner 10 is transitioned into the folded configuration.

The various panels described above may be joined by seams, using conventional seaming techniques, such as sewing, welding or the like. Furthermore, the materials listed above are exemplary in nature only and those skilled in the art will readily appreciate that other materials may be used without departing from the spirit and scope of the present invention.

According to one embodiment the brace liner 10 includes additional padding or protective elements connected thereto to provide additional protection to the wearer's knee. For instance, the brace liner 10 may include a knee pad 60 connected to the brace liner 10 for protecting the user's knee cap. In the exemplary embodiment, the knee pad 60 is an annular pad having an opening which is placed over the user's knee cap during use of the brace liner 10. The knee pad 60 is preferably connected (i.e., sewn) to the inner surface of the brace liner 10, although it is understood that the knee pad may also be connected to the outer surface thereof.

It is also contemplated that various embodiments of the brace liner 10 may include other padding or protective elements. For instance, the brace liner 10 may include padding positioned over the wearer's thigh and/or hip when the brace liner 10 is worn by the wearer. It is contemplated that the padding may be permanently secured to the brace liner 10 (i.e., sewn into the brace liner 10) or, alternatively, removable therefrom. In this regard, the removable padding may be placed within a pocket formed within the brace liner 10 during usage of the brace liner 10.

The brace liner 10 may also include a rear support element 62 which is disposable behind the wearer's knee during use and may further protect against hyperextension of the knee. The rear support element may define an “x” configuration and may be formed from a material which tightly conforms to the user's knee and provides support to the rear of the knee.

Referring now back to FIG. 1, those skilled in the art will appreciate that many conventional knee braces 12 include a proximal end portion 64, a distal end portion 66 and a bend region 68 disposed between the proximal end portion 64 and the distal end portion 66. The knee braces 12 may additionally include a knee cap guard 70 connected to the proximal end portion 64 and the distal end portion 66. As shown in FIGS. 1 and 2, an embodiment of the brace liner 10 is configured to extend over the distal end portion 66, knee cap guard 70 and proximal end portion 64 of the knee brace 10 when the brace liner 10 is transitioned into the folded configuration.

It is also contemplated that various embodiments of the brace liner 10 may be specifically configured for use with a knee brace 12 having a detached knee cap guard 70. In other words, the knee cap guard 70 may be separate and detached from the proximal end portion 64 and the distal end portion 66 and the brace liner 10 may be configured to maintain the detached knee cap guard 70 in proper position during usage of the brace 12. Referring now specifically to FIG. 5, one embodiment of the brace liner 10 includes a knee cap retention element 72 connected to the distal end portion 22, 24 of each leg portion 14, 16. The knee cap retention element 72 may include a mechanical fastener, such as hook and loop fasteners (i.e., VELCRO™), snaps, or the like, which engages with a complimentary fastener on the knee cap guard 70. In this regard, the convex surface of the knee cap guard 70 may include a mechanical fastener to couple the knee cap guard 70 to the knee cap retention element 72 on the brace liner 10. The knee cap retention element 72 may be positioned on the brace liner 10 such that when the knee cap guard 70 is connected to the retention element 72 and the brace liner 10 is in the folded configuration, the knee cap guard 70 is properly positioned over the user's knee cap between the proximal end portion 64 and the distal end portion 66 of the brace 12.

In use, the user initially dons the brace liner 10 on his hips/legs, as described above such that the brace liner 10 is in the unfolded configuration. The user would then connect the knee cap guard 70 to the retention element 72, and connect the brace 12 to the user's knee. Subsequently, the user would fold the brace liner 10 from the unfolded configuration to the folded configuration, which also places the knee cap guard 70 in proper position over the user's knee cap.

Additional modifications and improvements of the present invention may also be apparent to those of ordinary skill in the art. Thus, the particular combination of components and steps described and illustrated herein is intended to represent only certain embodiments of the present invention, and is not intended to serve as limitations of alternative devices and methods within the spirit and scope of the invention.

What is claimed is:

1. A liner for use with a brace disposable on a user's limb, the brace having a proximal end portion, a distal end portion and a bend region between the proximal and distal end portions, the liner comprising:
a first limb portion having:
a first surface and an opposing second surface, the first surface defining an opening sized to receive the user's limb; and
a first end portion, a second end portion, and a middle portion extending between the first end portion and the second end portion;
the first limb portion being transitional between an unfolded configuration and a folded configuration, the brace being disposable over the first limb portion when the first limb portion is in the unfolded configuration, the second end portion being folded toward the first end portion when the first limb portion transitions from the unfolded configuration toward the folded configuration to capture at least the distal end portion of the brace within the first limb portion.

2. The liner as recited in claim 1, wherein the first limb portion defines an annular fold as the first limb portion transitions from the unfolded configuration toward the folded configuration.

3. The liner as recited in claim 1, further comprising a waist portion connected to the first limb portion, the waist portion being wearable on the user's waist.

4. The liner as recited in claim 1, further comprising a slit formed within the first limb portion adjacent the second end portion and extending between the first surface and the second surface, the slit being configured to allow the first limb portion to extend about the distal end portion of the brace as the first limb portion transitions into the folded configuration.

5. The liner as recited in claim 1, wherein the first limb portion captures the distal end portion, bend region and proximal end portion of the brace when the first limb portion is in the folded configuration.

6. The liner as recited in claim 1, wherein middle portion of the first limb portion defines a convex portion and a concave portion, the liner further comprising a first pad coupled to the first surface adjacent the convex portion.

7. The liner as recited in claim 1, wherein middle portion of the first limb portion defines a convex portion and a concave portion, the liner further comprising a second pad coupled to the first surface adjacent the concave portion.

8. The liner as recited in claim 1, wherein at least a portion of the first limb portion is formed from a moisture wicking elastic material.

9. A liner for use with a first knee brace disposable on a first leg of a user and a second knee brace disposable on second leg of the user, each knee brace having a respective proximal end portion, a respective distal end portion and a respective bend region between the respective proximal and distal end portions, the liner comprising:
a first leg portion having:
a first surface and an opposing second surface, the first surface defining an opening sized to receive the user's first leg; and
a first end portion, a second end portion, and a middle portion extending between the first end portion and the second end portion;
the first leg portion being transitional between an unfolded configuration and a folded configuration, the first brace being disposable over the first leg portion when the first leg portion is in the unfolded configuration, the second end portion being folded toward the first end portion when the first limb portion transitions from the unfolded configuration toward the folded configuration to capture at least the distal end portion of the first brace within the first leg portion;
a second leg portion having:
a first surface and an opposing second surface, the first surface defining an opening sized to receive the user's second leg; and
a first end portion, a second end portion, and a middle portion extending between the first end portion and the second end portion;
the second leg portion being transitional between an unfolded configuration and a folded configuration, the second knee brace being disposable over the second leg portion when the second leg portion is in the unfolded configuration, the second end portion being folded toward the first end portion when the second leg portion transitions from the unfolded configuration toward the folded configuration to capture at least the distal end portion of the second knee brace within the second leg portion.

10. The liner as recited in claim 9, wherein the first leg portion defines an annular fold as the first leg portion transitions from the unfolded configuration toward the folded configuration.

11. The liner as recited in claim 10, wherein the second leg portion defines an annular fold as the second leg portion transitions from the unfolded configuration toward the folded configuration.

12. The liner as recited in claim 9, wherein the first leg portion is selectively transitional between the folded and unfolded configurations independent of the second leg portion.

13. The liner as recited in claim 9, further comprising a waist portion connected to the first and second leg portions, the waist portion being wearable on the user's waist.

14. The liner as recited in claim 9, wherein the first leg portion captures the distal end portion, bend region and proximal end portion of the first brace when the first leg portion is in the folded configuration.

15. The liner as recited in claim 9, wherein the second leg portion captures the distal end portion, bend region and proximal end portion of the second brace when the second leg portion is in the folded configuration.

16. The liner as recited in claim 9, further comprising a first slit formed within the first leg portion adjacent the second end portion thereof and extending between the first surface and the second surface, the first slit being configured to allow the first leg portion to extend about the distal end portion of the first knee brace as the first leg portion transitions into the folded configuration.

17. The liner as recited in claim 16, further comprising a second slit formed within the second leg portion adjacent the second end portion thereof and extending between the first surface and the second surface, the second slit being configured to allow the second leg portion to extend about the distal end portion of the second knee brace as the second leg portion transitions into the folded configuration.

18. A liner for use with a knee brace positionable on a wearer's leg, the knee brace having a proximal end portion, a distal end portion and a knee cap connected to the proximal and distal end portions, the liner comprising:
a first limb portion having:
a first surface and an opposing second surface, the first surface defining an opening sized to receive the user's limb; and
a first end portion, a second end portion, and a middle portion extending between the first end portion and the second end portion;
the first limb portion being transitional between an unfolded configuration and a folded configuration, the brace being disposable over the first limb portion when the first limb portion is in the unfolded configuration, the second end portion being folded toward the first end portion when the first limb portion transitions from the unfolded configuration toward the folded configuration to capture at least the distal end portion of the brace within the first limb portion; and
a knee cap connector coupled to the first limb portion and connectable to the knee cap to connect the knee cap to the liner, the knee cap connector being positioned on the first limb portion such that when the first limb portion is transitioned to the folded configuration, the knee cap is positioned between the proximal end portion and distal end portion of the knee brace.

19. The liner as recited in claim 18, wherein the first limb portion defines an annular fold as the first limb portion transitions from the unfolded configuration toward the folded configuration.

20. The liner as recited in claim 18, further comprising a waist portion connected to the first limb portion, the waist portion being wearable on the user's waist.
